(19) United States
(12) Patent Application Publication
(10) Pub. No.: US 2007/0122899 A1
(43) Pub. Date: May 31, 2007

(54) Target substance analysis apparatus and determination software update method

(75) Inventor: Yuri Mizutani, Yokohama-shi (JP)

Correspondence Address: FITZPATRICK CELLA HARPER & SCINTO, 30 ROCKEFELLER PLAZA, NEW YORK, NY, 10112, US

(73) Assignee: CANON KABUSHIKI KAISHA, Tokyo (JP)

(21) Appl. No.: 11/603,059

(22) Filed: Nov. 22, 2006

(30) Foreign Application Priority Data
Nov. 28, 2005 (JP) 2005-341919

(51) Int. Cl. C12M 1/34; A61L 9/01; G05B 21/02

(52) U.S. Cl. 435/287.2; 700/73; 435/266

(57) ABSTRACT

There is provided a biochemical reaction cassette analysis apparatus capable of automatically updating determination software. A biochemical-reaction-cassette-equipped automatic analysis apparatus having a storage section in which determination software is stored in association with its version information and identification information for identifying the kind of probe on a probe carrier is connected to a determination software provision system via a network. The determination software provision system has a determination software information storage section in which determination software corresponding to the kind of the probe is stored in association with its version information and the identification information and a checking computer which provides update information of the determination software in the determination software information storage section to the biochemical-reaction-cassette-equipped automatic analysis apparatus.

### BACKGROUND OF THE INVENTION

1. Field of the Invention

The present invention relates to an analysis apparatus for determining the kind and the amount of a target substance with the use of a probe carrier configured by fixing a probe (a labeled biochemical substance) specifically bindable to the target substance and included in a biochemical reaction cassette or the like, and a method for updating determination software used therefor.

2. Description of the Related Art

With the increase of genomic information, research for examining the polymorphism and the expression level of genes is promoted at a large scale. As an example of a probe carrier often used for high throughput genome analysis, there is a DNA microarray (also referred to as a DNA chip). The DNA microarray is configured by fixing a probe specifically bindable to a target substance, such as DNA, on a glass slide at a high density. A gene is extracted from a specimen, such as obtained blood, and amplified on this glass slide on which the probe is fixed at a high density, and hybridization reaction of a genome labeled with fluorescent dye or the like is caused. If a target substance exists in the specimen, the labeled target substance is bound to the probe, and thereby the kind and the amount of the target substance are known from the position and the amount of the label (see Japanese Patent Application Laid-Open No. H11-187900).

A DNA microarray analysis system commonly used now is configured by multiple apparatuses such as an extraction apparatus, an amplification apparatus, a labeling apparatus, a hybridization apparatus, a detection apparatus and an analysis apparatus. The extraction apparatus extracts a gene from a specimen. The amplification apparatus amplifies the extracted gene. The labeling apparatus labels the amplified gene with fluorescent dye. The hybridization apparatus hybridizes a genome created by the processes at the extraction apparatus, the amplification apparatus and the labeling apparatus to a DNA microarray carrier. The detection apparatus detects the position and the amount of the fluorescent label on the DNA microarray after reaction. The analysis apparatus analyzes the result detected by the detection apparatus. It is troublesome to move the specimen, the DNA microarray and the like among the apparatuses because the movement work is manually done. Furthermore, much space is required to place all the apparatuses.

Because the analysis system has been used mainly for research purposes, the troublesomeness of the manual work and the space problem have not been considered so much. However, since it is expected that the analysis system is also used for clinical purposes because of the good performance of the DNA microarray, it is necessary to consider such problems. Accordingly, there has been developed a biochemical-reaction-cassette-equipped automatic analysis apparatus in which the various apparatuses described above are integrated.

### SUMMARY OF THE INVENTION

As described above, there has been developed a biochemical-reaction-cassette-equipped automatic analysis apparatus in which various apparatuses are integrated. However, this biochemical-reaction-cassette-equipped automatic analysis apparatus presents problems as shown below when a user uses it.

As the technique for a method of designing a probe on a DNA microarray is improved and the research for correlation between genes and illness is advanced, the number of the kinds of probe used for a DNA microarray changes or increases. Furthermore, the pattern of the kinds of probe on a DNA microarray changes according to the content of testing (for example, combinations of cancer and infections, cancer and adverse effect of anticancer drug, and the like), it is conceivable that various chip patterns are thrown in. In such a situation, it may happen that, after a user purchases a biochemical-reaction-cassette-equipped automatic analysis apparatus, a biochemical reaction cassette including a DNA microarray with a new kind of probe is sold. In such a case, it may be impossible to perform analysis with the use of the DNA microarray with a new kind of probe sold after the purchase of the apparatus, by the determination software for determining the amount and the kind of a target substance, which was installed when the apparatus was purchased. Therefore, when purchase of the DNA microarray with a new kind of probe, the user has to acquire the latest determination software for performing analysis with the use of the purchased DNA microarray from the vendor and install it in the apparatus. However, it is very troublesome for the user to install the latest determination software in the biochemical-reaction-cassette-equipped automatic analysis apparatus by oneself.

An object of the present invention is to solve the above problem and provide a target substance analysis apparatus which does not impose a heavy burden on a user and which is capable of automatically updating determination software to the latest one.

Another object of the present invention is to provide a determination software update system and a determination software update method capable of making more precise determination.

Still another object of the present invention is to provide a method of operation of a target substance analysis apparatus, which is capable of performing qualitative and quantitative analysis of a target substance in a specimen at a higher precision.

That is, the present invention is a biochemical reaction cassette analysis apparatus for performing analysis with the use of a biochemical reaction cassette including a probe carrier on which a probe specifically bindable to a target substance is fixed, the analysis apparatus comprising, an information reading section which reads, from an information medium from which probe identification information for identifying information about the probe in the biochemical reaction cassette is readable, the probe identification information, a storage section in which determination software for analyzing the target substance is stored in association with version information about the determination software and the probe identification information, and a control section which acquires determination software from the storage section based on the probe identification information read by the information reading section to use the determination software for analysis of the target substance, wherein when update information of determination software including the version information and the probe identification information is provided from the outside, the control section updates information of appropriate determination software based on the version information and the probe kind identification information in the provided update information.

According to the present invention, an advantage is obtained that, determination software is automatically updated to the latest one, and a user does not have to perform operations or make settings for installing the latest determination software by oneself, and therefore a burden imposed on the user is reduced.

Furthermore, another advantage is obtained that automatic update eliminates a human-caused mistake, and as a result, the accuracy of testing/analysis is improved.

Other features and advantages of the present invention will be apparent from the following description taken in conjunction with the accompanying drawings, in which like reference characters designate the same or similar parts throughout the figures thereof.

### BRIEF DESCRIPTION OF THE DRAWINGS

FIG. 1 is a block diagram showing the configuration of an automatic DNA-determination-software update system which is a first embodiment of the present invention.

FIG. 2 is a block diagram showing the configuration of a determination software information management section of a biochemical reaction cassette analysis apparatus shown in FIG. 1.

FIG. 3 is a flowchart showing a procedure for DNA determination software update processing performed in the automatic DNA-determination-software update system shown in FIG. 1.

FIG. 4 is a schematic diagram showing the flow of data in the DNA determination software update processing shown in FIG. 3.

FIG. 5 is a flowchart showing a procedure for testing/analysis data collection processing performed in the automatic DNA-determination-software update system shown in FIG. 1.

FIG. 6 is a schematic diagram showing the flow of data in the testing/analysis data collection processing shown in FIG. 5.

FIG. 7 is a block diagram showing the configuration of an automatic DNA-determination-software update system which is a second embodiment of the present invention.

FIG. 8 is a flowchart showing a procedure for DNA determination software update processing performed in the automatic DNA-determination-software update system shown in FIG. 7.

FIG. 9 is a flowchart showing a procedure for testing/analysis processing with the use of determination software performed in the biochemical reaction cassette analysis apparatus shown in FIG. 8.

### DESCRIPTION OF THE EMBODIMENTS

Preferred embodiments of the present invention will now be described in detail in accordance with the accompanying drawings.

An aspect of the biochemical reaction cassette analysis apparatus according to the present invention is characterized in that there are provided a biochemical reaction cassette including a probe carrier on which a probe specifically bindable to a target substance is fixed together with an information medium from which probe kind identification information for identifying the kind of the probe is readable; a storage section in which determination software for analyzing the target substance is stored in association with version information about the determination software and the probe kind identification information; an information reading section which reads the probe kind identification information from the information medium; and a control section which acquires determination software from the storage section based on the probe kind identification information read by the information reading section to use the determination software for analysis of the target substance; and that, when update information of determination software including the version information and the probe kind identification information is provided from the outside, the control section searches the storage section based on the version information and the probe kind identification information in the provided update information to update information of appropriate determination software.

According to the above configuration, determination software stored in the storage section (installed determination software) is managed based on version information and probe kind identification information. When the latest determination software is provided from the outside, appropriate determination software in the storage section is automatically updated based on the version information and the probe kind identification information about the provided latest determination software. The determination software is acquired from the storage section based on the probe kind identification information read by the information reading section, and thereby, it is possible to use updated determination software. Therefore, it is not necessary for a user to perform operations for installing the latest determination software or make settings for using the installed determination software by oneself.

The target substance analysis apparatus according to the present invention is an analysis apparatus for a target substance which is to be used together with a probe carrier on which a probe specifically bindable to a target substance is fixed together with an information medium from which probe kind identification information for identifying the kind of the probe is readable, the analysis apparatus comprising:

(i) a storage section in which determination software for analyzing the target substance is stored in association with version information of the determination software and the probe kind identification information;

(ii) an information reading section which reads the probe kind identification information, from the information medium; and

(iii) a control section, wherein the control section executes the steps of, acquiring determination software from the storage section based on the probe kind identification information read by the information reading section, and when update information of determination software including the version information and the probe kind identification information is provided from the outside, searching the storage section based on the version information and the probe kind identification information in the provided update information to update information of appropriate determination software.

The target substance analysis apparatus according to the present invention is an analysis apparatus for a target substance which is to be used together with a probe carrier on which a probe specifically bindable to a target substance is fixed together with an information medium from which probe kind identification information for identifying the kind of the probe is readable, the analysis apparatus comprising, a signal detection section for detecting a signal from the probe carrier, a storage section in which determination software for analyzing the target substance is stored in association with version information about the determination software and the probe kind identification information, an information reading section which reads the probe kind identification information from the information medium, and a control section, wherein the control section executes the steps of:

(i) causing the signal detection section to detect a signal from the probe carrier provided for reaction with a specimen having a possibility of including the target substance;

(ii) acquiring corresponding determination software from the information medium fixed on the probe carrier mounted on a mounting section, based on the probe kind identification information read by the information reading section;

(iii) requesting update of the determination software acquired at the step (ii); and

(iv) if updated determination software is provided in response to the update request at the step (iii), analyzing the signal detected at the step (i) with the use of the updated determination software.

The method of operation of the target substance analysis apparatus according to the present invention is a method of operation of an analysis apparatus for a target substance which is to be used together with a probe carrier on which a probe specifically bindable to a target substance is fixed together with an information medium from which probe kind identification information for identifying the kind of the probe is readable, the analysis apparatus comprising, a signal detection section for detecting a signal from the probe carrier, a storage section in which determination software for analyzing the target substance is stored in association with version information about the determination software and the probe kind identification information, an information reading section which reads the probe kind identification information from the information medium, and a control section, wherein the control section executes the steps of:

(i) causing the signal detection section to detect a signal from the probe carrier provided for reaction with a specimen having a possibility of including the target substance;

(ii) acquiring corresponding determination software from the information medium fixed on the probe carrier mounted on a mounting section, based on the probe kind identification information read by the information reading section;

(iii) requesting update of the determination software acquired at the step (ii); and

(iv) if updated determination software is provided in response to the update request at the step (iii), analyzing the signal detected at the step (i) with the use of the updated determination software.

Embodiments of the present invention will be described below in detail.

### First Embodiment

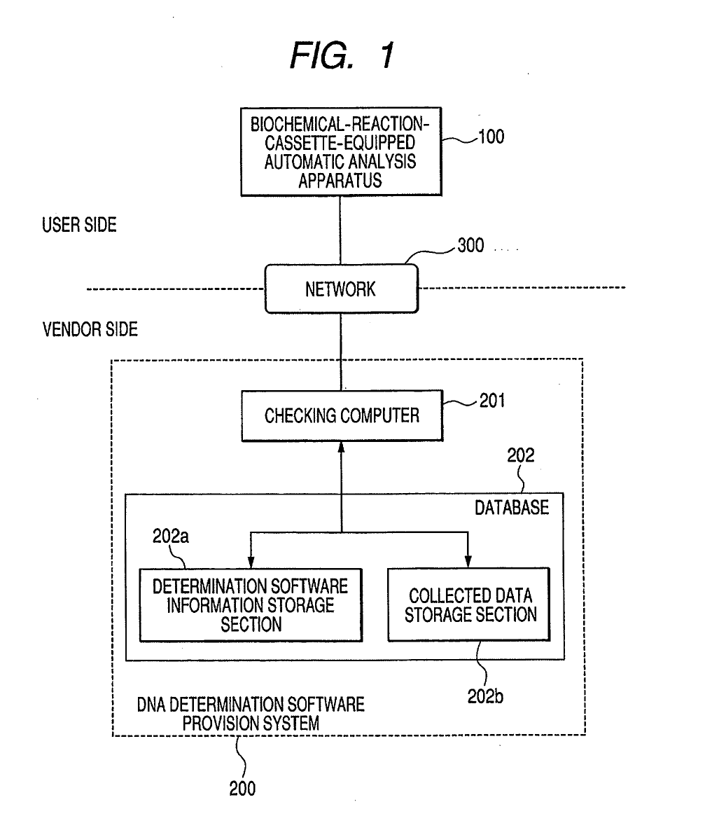

FIG. 1 is a block diagram showing the configuration of an automatic DNA-determination-software update system which is a first embodiment of the present invention. Referring to FIG. 1, the automatic DNA-determination-software update system is configured by a biochemical-reaction-cassette-equipped automatic analysis apparatus 100 used on the user side and a DNA determination software provision system 200 used on the vendor side. The biochemical-reaction-cassette-equipped automatic analysis apparatus 100 and the DNA determination software provision system 200 are connected via a network 300 so that they can mutually communicate with each other.

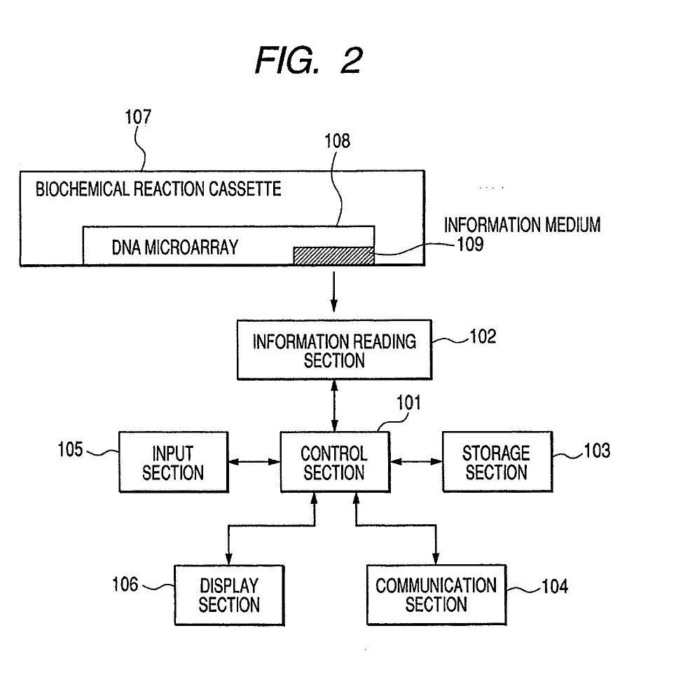

The biochemical-reaction-cassette-equipped automatic analysis apparatus 100 is configured by integrating an extraction apparatus, an amplification apparatus, a labeling apparatus, a hybridization apparatus, a detection apparatus, an analysis apparatus and the like, and it is provided with a determination software information management section. FIG. 2 shows the configuration of the determination software information management section.

Referring to FIG. 2, the determination software information management section is configured by an information reading section 102, a storage section 103, a communication section 104, an input section 105, a display section 106 and a control section 101 which controls the operation of each of these sections. The communication section 104 is a modem or the like which communicates with an external apparatus through the network 300. The input section 105 includes a device, such as a keyboard and a mouse, which is used when a user inputs data, a disk device for reading information from a recording medium such as a CD-ROM and a DVD, and the like. The display section 106 is configured by a CRT or an LCD.

The information reading section 102 reads information from an information medium 109 provided at a part of a microarray 108 included in a biochemical reaction cassette 107. The information medium 109 is a medium in which identification information (for example, a code) capable of identifying information about a probe on the microarray 108 (for example, information about the kind, the amount and the arrangement of the probe) is recorded (for example, printed) or stored. As the information medium 109, a wireless tag (an IC chip) in which probe kind identification information is stored is included, in addition to the medium on which the probe kind identification information is printed as barcode information or image information. When barcode information is used, a barcode reader is used as the information reading section 102. When image information is used, an image sensor configured by an image-pickup device represented by a CCD is used as the information reading section 102. When a wireless tag is used, a reader for wireless tag is used as the information reading section 102. Attachment of the information medium 109 to the microarray 108 is performed by the vendor side.

The target to which the information medium is attached is not limited to the microarray 108. The information medium may be attached to the biochemical reaction cassette 107 itself. Furthermore, it may be attached to a card or the like provided by the vendor side. The card may be inserted into the biochemical-reaction-cassette-equipped automatic analysis apparatus 100 to check the identification information.

In the storage section 103, there are stored determination software information, a program for managing the determination software information and data (testing/analysis data), information required for access to the DNA determination software provision system 200 and the like. The determination software information is information of the determination software already installed at the purchase of the analysis apparatus or the determination software installed by the user side with the use of the input section 105 after the purchase of the analysis apparatus. This determination software information includes version information indicating the degree of newness of the software and identification information about the usable kind of probe. The installation processing is executed by the control section 101. The information required for access includes address information about each of the DNA determination software provision system 200 and the automatic analysis apparatus 100 on the network 300, identification information about the automatic analysis apparatus 100 and the like.

The control section 101 operates in accordance with the program stored in the storage section 103, and it controls reading of the probe kind identification information by the information reading section 102 as well as searching the storage section 103 to acquire determination software information, based on the read probe kind identification information. The acquired determination software is used for analysis. The control section 101 accesses the DNA determination software provision system 200 based on the address information stored in the storage section 103 and makes a software update request. When making the software update request, the control section 101 sends version information about the detected determination software information and the probe kind identification information, from the communication section 104 to the DNA determination software provision system 200.

Any software can be used as the determination software if it is capable of determining the existence or the abundance of a target substance from the testing result, based on information about a probe carrier to be used (the kind of, the arrangement information about, and the abundance of the probe).

For example, software which compares testing data with similar testing data accumulated in the past and identifies the most similar data to determine the existence of a target substance (determination of a particular infection bacteria, SNP determination and the like) is given as an example. Software for determining an abnormal abundance of a target substance when a numerical parameter obtained from testing exceeds a threshold (such as determination of the expression level of mRNA) is also given as an example.

In particular, in the case of using a DNA microarray as a probe carrier, a signal (such as a fluorescent luminance value) acquired from the array can be obtained as two-dimensional image information by reacting a target DNA labeled with fluorescent material with the microarray.

A method of making determination by comparing the obtained two-dimensional image information with two-dimensional image information to be determined, by a pattern matching method or the like may be adopted.

Description is made on more specific determination software in Japanese Patent Application Laid-Open No. 2005-003676, and the software can be used in the present invention.

When receiving the latest software from the DNA determination software provision system 200, the control section 101 updates appropriate software in the storage section 103 with the received latest software. The latest software is, for example, such that the brightness threshold for each probe kind or the value of fluctuation of the brightness pattern of the entire probe has been changed. When having executed testing/analysis processing with the use of the microarray 108 included in the biochemical reaction cassette 107, the control section 101 sends data of the testing/analysis from the communication section 104 to the DNA determination software provision system 200. The testing/analysis data may be sent in response to a collection request by the DNA determination software provision system 200. In this case, the testing/analysis data is stored in the storage section 103 in association with information of determination software (including version information and probe kind identification information) until a collection request is received.

The DNA determination software provision system 200 is configured by a checking computer 201 and a database 202. The database 202 has a determination software information storage section 202a and a collected data storage section 202b. In the determination software information storage section 202a , the latest determination software information corresponding to the kind of each probe on the DNA microarray 108 is stored. This latest determination software information includes version information and identification information about the kinds of probe which can be used. In the collected data storage section 202b, data of testing/analysis collected from the biochemical-reaction-cassette-equipped automatic analysis apparatus 100 is stored in association with information of the determination software used for the testing/analysis (including version information and the kind of probe).

The checking computer 201 is provided with an input device such as a keyboard and a mouse, a storage section in which programs and data are stored, a communication function of communicating with an external apparatus via the network 300 and the like. In response to a software update request by the biochemical-reaction-cassette-equipped automatic analysis apparatus. 100, the checking computer 201 searches the determination software information storage section 202a to check whether or not the requested latest determination software information exists. In the search of the determination software information storage section 202a, version information about the determination software and probe kind identification information received together with the software update request are used. If the latest determination software information exists, then the checking computer 201 sends the determination software information to the biochemical-reaction-cassette-equipped automatic analysis apparatus 100. If the latest determination software information does not exist, then the checking computer 201 sends the notification to that effect to the biochemical-reaction-cassette-equipped automatic analysis apparatus 100. The checking computer 201 also collects testing/analysis data and information of the determination software used for the testing/analysis (including version information and the probe) from the biochemical-reaction-cassette-equipped automatic analysis apparatus 100 which has accessed the checking computer 201. The data collection may be periodically performed. Alternatively, after performing testing/analysis processing, the biochemical-reaction-cassette-equipped automatic analysis apparatus 100 may automatically send data of the testing/analysis. The collected data is stored in the collected data storage section 202b in association with information of the determination software (including version information and the kind of probe).

A vendor (specifically, a system operation administrator) can update determination software information in the determination software information storage section 202a to the latest information by performing an input operation for data update on the input device of the checking computer 201. In the information update, the vendor can use collected data stored in the collected data storage section 202b.

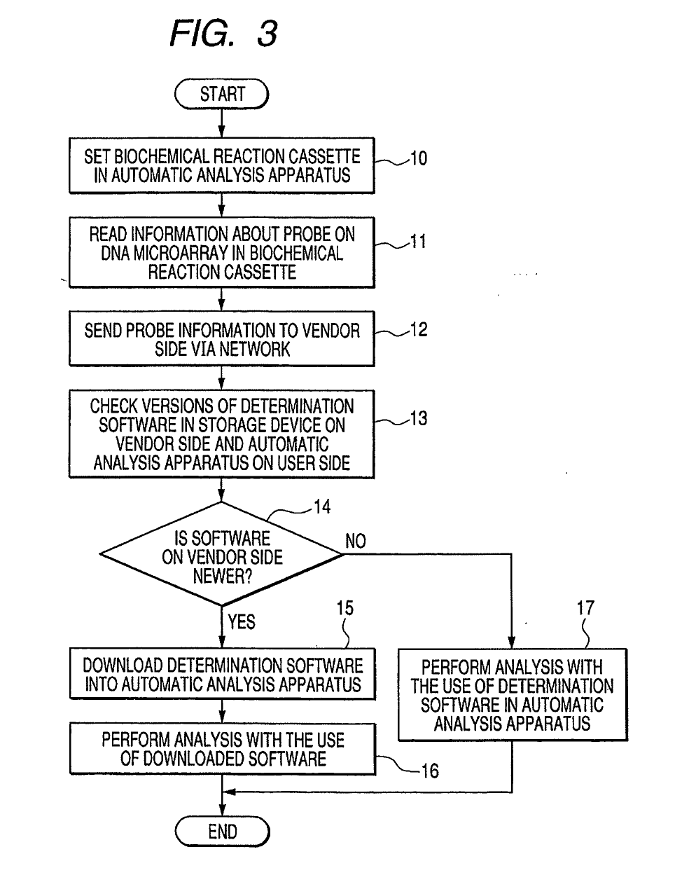

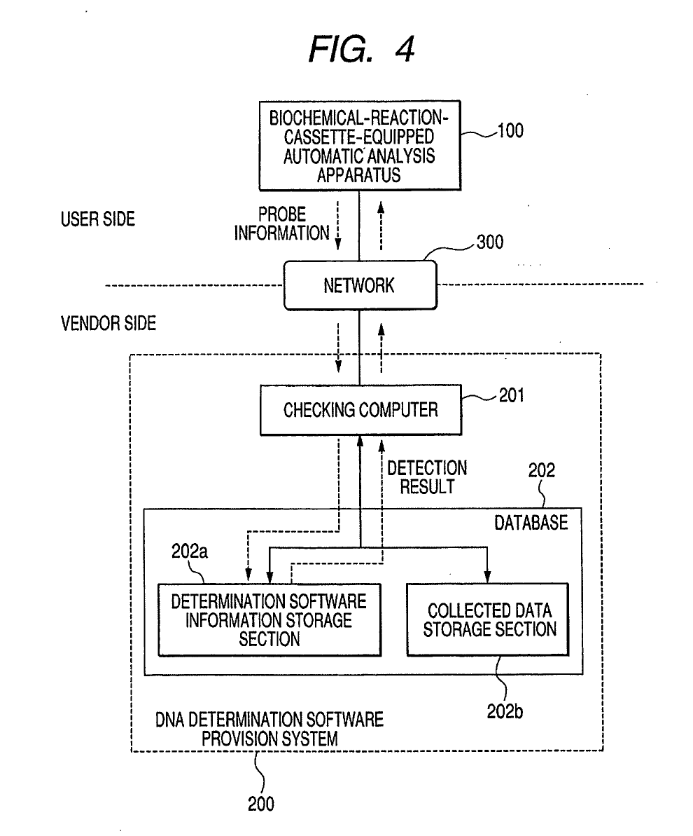

Next, the operation of the automatic DNA-determination-software update system of this embodiment will be specifically described. FIG. 3 is a flowchart showing a procedure for the DNA determination software update processing, and FIG. 4 is a schematic diagram showing the flow of data in the DNA determination software update. The DNA determination software update processing will be specifically described below with reference to FIGS. 1 to 4.

A user sets any of a biochemical reaction cassette 107 in which a microarray 108 has been already included by the vendor and a biochemical reaction cassette 107 in which a microarray 108 purchased from the vendor in advance is included by the user, into the biochemical-reaction-cassette-equipped automatic analysis apparatus 100 (step 10). When detecting that the biochemical reaction cassette 107 has been set, the control section 101 controls the information reading section 102 to read probe kind identification information from an information medium 109 provided for the microarray 108 (step 11). Then, the control section 101 searches the storage section 103 to acquire determination software information, based on the read probe kind identification information.

After acquiring the determination software information, the control section 101 accesses the DNA determination software provision system 200 based on the address information stored in the storage section 103 and makes a software update request (step 12). At this software update request sending step, the control section 101 sends probe information which includes version information about the retrieved determination software information and probe kind identification information, to the DNA determination software provision system 200 from the communication section 104.

In the DNA determination software provision system 200, the checking computer 201 searches the determination software information storage section 202a based on the probe information received from the biochemical-reaction-cassette-equipped automatic analysis apparatus 100. Then, the checking computer 201 checks the version of the: determination software included in the received probe information against the version of the retrieved determination software (step 13). Then, the checking computer 201 determines whether or not the determination software on the vendor side is newer based on the checking result (step 14). At this determination, the determination software with a larger version number is assumed to be newer.

If the determination at step 14 is “Yes”, then the checking computer 201 sends information of the determination software (including the probe kind identification information and the version information) to the biochemical-reaction-cassette-equipped automatic analysis apparatus 100 as the latest information. In the biochemical-reaction-cassette-equipped automatic analysis apparatus 100 which has received information of the latest determination software, the control section 101 stores the information of the latest determination software in the storage section 103 (step 15). In this storage (download) processing, the control section 101 rewrites the older-version determination software with the newer-version determination software, based on the version information and the probe kind identification information. After the storage (download) processing, analysis with the use of the newer-version determination software is performed in the biochemical-reaction-cassette-equipped automatic analysis apparatus 100 (step 16).

If the determination at step 14 is “No”, then the checking computer 201 sends a notification signal indicating that the determination software currently used is the latest one to the biochemical-reaction-cassette-equipped automatic analysis apparatus 100. In the biochemical-reaction-cassette-equipped automatic analysis apparatus 100, the control section 101 determines that the determination software stored in the storage section 103 is the latest one, by receiving the notification signal. Accordingly, in the biochemical-reaction-cassette-equipped automatic analysis apparatus 100, analysis with the use of the determination software stored in the storage section 103 is performed (step 17). When this processing is: performed, the control section 101 which has received the notification signal may display a message to the effect that the determination software currently used is the latest one, on the display section 106.

In the system of this embodiment, in addition to the DNA determination software update processing described above, processing for collecting testing/analysis data from the biochemical-reaction-cassette-equipped automatic analysis apparatus 100 is also performed in the DNA determination software provision system 200 in order to improve the testing precision.

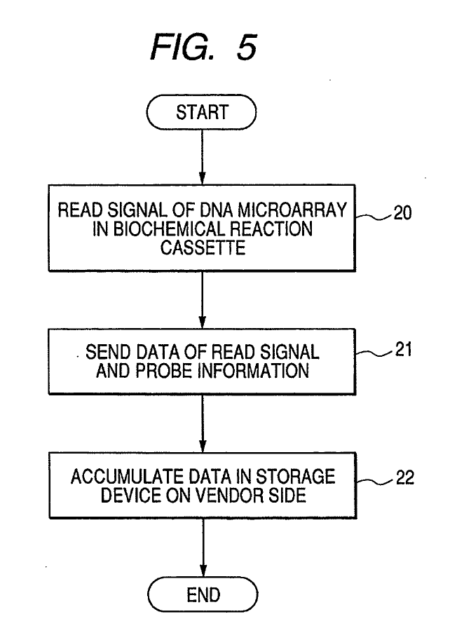

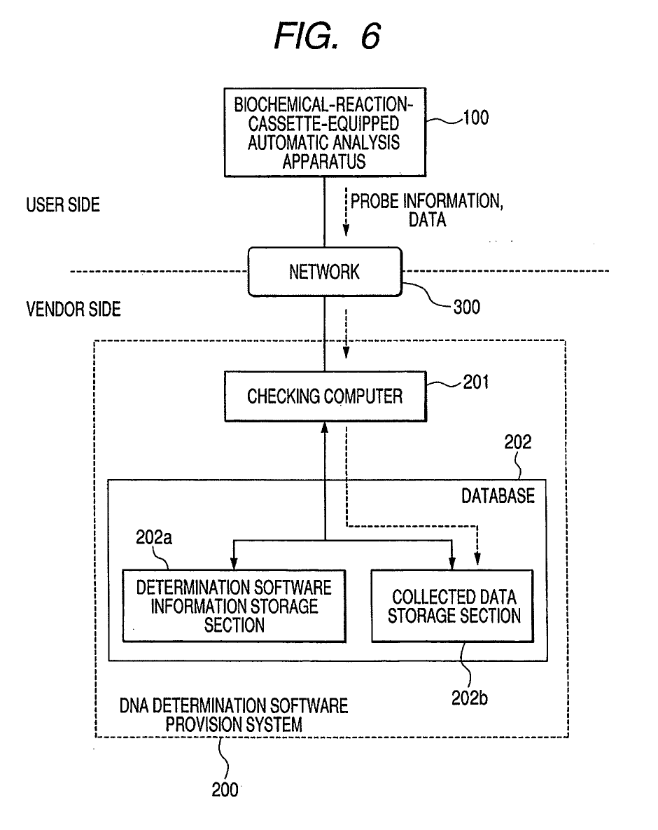

FIG. 5 is a flowchart showing a procedure for the testing/analysis data collection processing, and FIG. 6 is a schematic diagram showing the flow of data in the testing/analysis data collection. The testing/analysis data collection processing will be specifically described below with reference to FIGS. 1, 2, 5 and 6.

In the biochemical-reaction-cassette-equipped automatic analysis apparatus 100, the testing/analysis processing is executed for the microarray 108 in the set: biochemical reaction cassette 107, (step 20). In this processing, a gene is extracted from a specimen and amplified first, and then, a genome is created by labeling the amplified gene with fluorescent dye. Then, the created genome is hybridized. The position and the amount of the fluorescent label on the DNA microarray after reaction are detected, and the detection result is analyzed.

After the testing/analysis processing, the control section 101 searches the storage section 103 to acquire determination software information, based on the probe kind identification information read from the information medium 109 by the information reading section 102. Then, the control section 101 accesses the DNA determination software provision system 200 based on the address information stored in the storage section 103. Then, the control section 101 sends testing/analysis data to the DNA determination software provision system 200 together with the probe information including the determination software information (including the version information and the probe kind identification information) (step 21).

In the DNA determination software provision system 200 which has received the testing/analysis data and the probe information, the checking computer 201 stores the received testing/analysis data and probe information in the collected data storage section 202b (step 22).

According to the embodiment described above, at the timing when the biochemical reaction cassette 107 is set, update to the latest determination software corresponding to the microarray 108 included in the cassette is automatically performed. Therefore, it is not necessary for the user to perform operations or settings to update the software by oneself.

Furthermore, since determination software is always updated to the latest determination software, it is also not necessary for the user to always grasp the latest determination software corresponding to the kind of probe by oneself. Conventionally, such configuration has not been provided, and it is necessary for the user to always grasp information about the latest determination software by oneself.

In the case of using the analysis system for clinical purposes, the testing result is required to be highly accurate because a human life is involved. According to this embodiment, since update is automatically performed, a human-caused mistake is eliminated. As a result, the accuracy of testing/analysis is improved, and it is possible to meet the request for the accuracy in the clinical use.

Furthermore, in this embodiment, a lot of data obtained by testing with the use of DNA microarrays, on which the same kind of probe is fixed, are collected from a user, and the latest determination software is created based on the large amount of collected data. Thereby, the testing precision can be improved.

### Second Embodiment

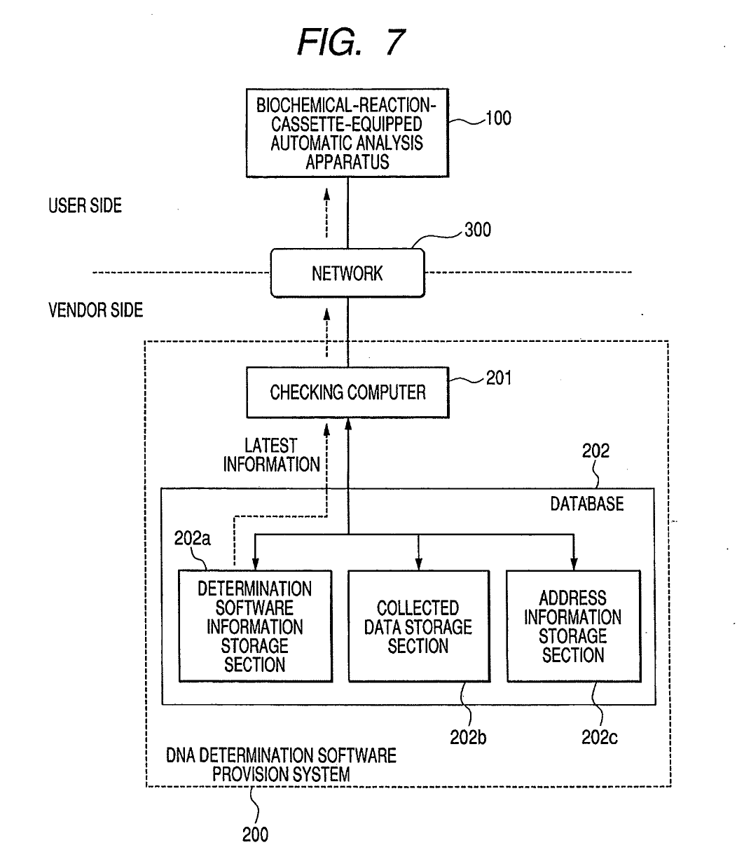

FIG. 7 is a block diagram showing the configuration of an automatic DNA-determination-software update system which is a second embodiment of the present invention. The automatic DNA-determination-software update system of this embodiment is different from the system of the first embodiment in the operation of providing information of the latest determination software by the checking computer 201. In the first embodiment, the biochemical-reaction-cassette-equipped automatic analysis apparatus 100 accesses the DNA determination software provision system 200 to request for update to the latest determination software at the timing when the biochemical reaction cassette 107 is set. In this embodiment, on the contrary, every time software information is updated at the determination software information storage section 202a, the DNA determination software provision system 200 automatically sends the update information to the biochemical-reaction-cassette-equipped automatic analysis apparatus 100. Since the configurations other than this are similar to those in the system of the first embodiment, only the different part will be described below in detail.

As shown in FIG. 7, in the automatic DNA-determination-software update system of this embodiment, the database 202 has an address information storage section 202c in addition to the determination software information: storage section 202a and the collected data storage section 202b. In the address information storage section 202c, the address information on the network 300, of a biochemical-reaction-cassette-equipped automatic analysis apparatus to which information of updated determination software is to be sent is stored in advance. As for the registration of the address information with the address information storage section 202c, address information presented by a user is registered by a system administrator when the analysis apparatus is purchased. This address information registration may be performed by accessing the DNA determination software provision system 200 from the biochemical-reaction-cassette-equipped automatic analysis apparatus 100 after purchasing the biochemical-reaction-cassette-equipped automatic analysis apparatus 100.

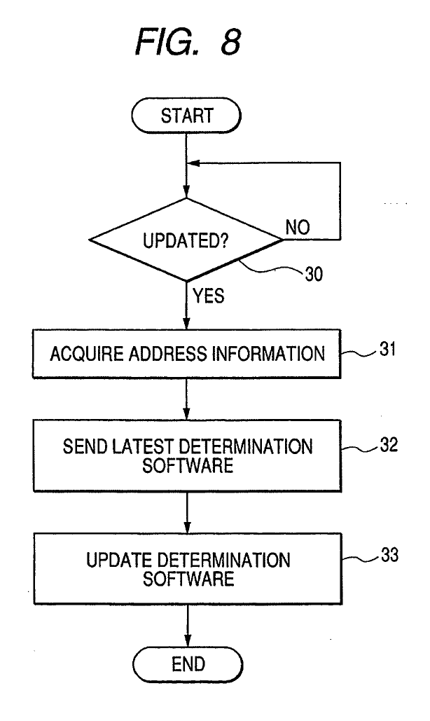

FIG. 8 shows a procedure for the DNA determination software update processing performed in the automatic DNA-determination-software update system which is the second embodiment of the present invention. The DNA determination software update processing will be described below with reference to FIGS. 7 and 8.

On the checking computer 201, a system operation administrator performs an input operation for updating determination software stored in the determination software information storage section 202a. In response to this input operation, the checking computer 201 determines that the determination software has been updated (step 30: Yes). When the determination software is updated, the checking computer 201 acquires address information about a biochemical-reaction-cassette-equipped automatic analysis apparatus to which information of the updated determination software is to be sent, from the address information storage section 202c (step 31). Then, the checking computer 201 acquires the information of the updated determination software (including version information and probe kind identification information) from the determination software information storage section 202a and sends it to the address information acquired at step 31 (step 32).

In the biochemical-reaction-cassette-equipped automatic analysis apparatus 100, the control section 101 updates the information stored in the storage section 103 based on the information of the updated determination software received from the checking computer 201 (step 33). In this information update, the storage section 103 is searched based on the version information and the probe kind identification information included in the information of the updated determination software. Then, information of appropriate determination software is rewritten with the information of the updated determination software.

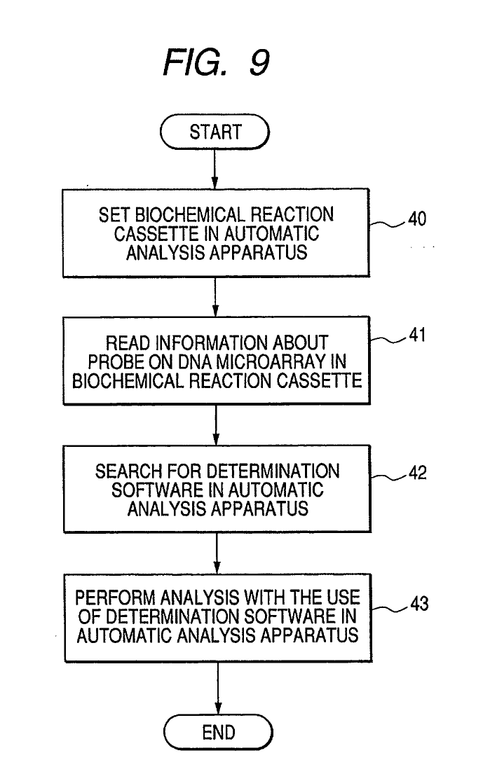

After information of the determination software in the storage section 103 is updated as described above, testing/analysis with the use of the determination software in the storage section 103 is performed in the biochemical-reaction-cassette-equipped automatic analysis apparatus 100. FIG. 9 shows a procedure for the testing/analysis processing with the use of the determination software.

Referring to FIG. 9, the user first sets a biochemical reaction cassette 107 in which a microarray 108 has been already included by the vendor or a cassette in which a microarray 108 purchased from the vendor in advance is included, into the biochemical-reaction-cassette-equipped automatic analysis apparatus 100 (step 40). When detecting that the biochemical reaction cassette 107 has been set, the control section 101 controls the information reading section 102 to read probe kind identification information from the information medium 109 provided for the microarray 108 (step 41). Then, the control section 101 searches the storage section 103 to read appropriate determination software information, based on the read probe kind identification information (step 42). In the biochemical-reaction-cassette-equipped automatic analysis apparatus 100, analysis with the use of the read determination software is performed (step 43).

In this embodiment also, the result of the testing/analysis by the biochemical-reaction-cassette-equipped automatic analysis apparatus 100 is collected by the DNA determination software provision system 200. Thereby, the vendor side can create the latest determination software based on the collected data.

According to this embodiment describe above, every time the information of the determination software in the determination software information storage section 202a is updated, the update information is provided for the biochemical-reaction-cassette-equipped automatic analysis apparatus 100 from the DNA determination software provision system 200. Then, in the biochemical-reaction-cassette-equipped automatic analysis apparatus 100, the information of the determination software in the storage section 103 is updated based on the update information. Accordingly, in this embodiment also, advantages similar to those of the first embodiment can be obtained.

In the first embodiment, update to the latest determination software is performed at the timing when a biochemical reaction cassette is set. In this embodiment, on the contrary, the latest determination software corresponding to the kind of probe is provided for the biochemical-reaction-cassette-equipped automatic analysis apparatus 100 in advance from the DNA determination software provision system 200. Therefore, when a biochemical reaction cassette is set, update to the latest determination software has already been performed, and it is possible to immediately execute analysis with the use of the latest determination software. Thus, in this embodiment, the processing time required after setting a biochemical reaction cassette is shortened by the time required for download, in comparison with the first embodiment.

Each of the embodiments described above is only an example of the present invention, and the configuration and the operation can be changed as appropriate. For example, the configurations of the first embodiment and second embodiments can be combined. In this case, the determination software update processing in the biochemical-reaction-cassette-equipped automatic analysis apparatus 100 is executed at the timing when a biochemical reaction cassette is set. In addition to this update processing, the determination software update processing in the biochemical-reaction-cassette-equipped automatic analysis apparatus 100 is executed at the time when information of determination software stored in the determination software information storage section 202a is updated.

In the system configuration shown in FIGS. 1 and 4, one biochemical-reaction-cassette-equipped automatic analysis apparatus 100 is connected to the DNA determination software provision system 200. However, the present invention is not limited thereto. The number of the biochemical-reaction-cassette-equipped automatic analysis apparatuses 100 connected to the DNA determination software provision system 200 may be 2 or more. Commonly, multiple biochemical-reaction-cassette-equipped automatic analysis apparatuses 100 are connected to the DNA determination software provision system 200.

The update information of determination software is sent to the biochemical-reaction-cassette-equipped automatic analysis apparatus 100 from the DNA determination software provision system 200 via the network 300. However, the present invention is not limited thereto. For example, the update information of determination software may be provided for the biochemical-reaction-cassette-equipped automatic analysis apparatus 100 through a recording medium such as a CD-ROM.

The microarray 108 is given as an example of the probe carrier to be included in the biochemical reaction cassette 107. However, the present invention is not limited thereto. The probe carrier may be configured in any form if a probe specifically bindable to a target substance is fixed thereon. As the probe, various substances such as nucleic acid and protein can be used.

The material of the microarray 108 may be glass or plastic.

An ink-jet method and a pin method may be used to fix a probe onto the microarray 108.

A flow channel may be formed in the biochemical reaction cassette 107. In the biochemical-reaction-cassette-equipped automatic analysis apparatus, not only an apparatus for qualitatively and/or quantitatively reading signals of a biochemical reaction cassette and a target substance and an apparatus for analyzing the signals but also various reaction fields, various processing fields, various fluid moving means and the like may be set.

The present invention is not limited to the above embodiments and various changes and modifications can be made within the spirit and scope of the present invention. Therefore to apprise the public of the scope of the present invention, the following claims are made.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2005-341919, filed Nov. 28, 2005, which is hereby incorporated by reference herein in its entirety.

What is claimed is:

1. A biochemical reaction cassette analysis apparatus for performing analysis with the use of a biochemical reaction cassette including a probe carrier on which a probe specifically bindable to a target substance is fixed, the analysis apparatus comprising:
an information reading section which reads, from an information medium from which probe identification information for identifying information about the probe in the biochemical reaction cassette is readable, the probe identification information;
a storage section in which determination software for analyzing the target substance is stored in association with version information about the determination software and the probe identification information; and
a control section which acquires determination software from the storage section based on the probe identification information read by the information reading section to use the determination software for analysis of the target substance; wherein
when update information of determination software including the version information and the probe identification information is provided from the outside, the control section updates information of appropriate determination software based on the version information and the probe identification information in the provided update information.

2. The biochemical reaction cassette analysis apparatus according to claim 1, wherein the probe identification information is probe kind identification information for identifying the kind of probe.

3. The biochemical reaction cassette analysis apparatus according to claim 1, wherein a biochemical reaction cassette with the information medium fixed on the probe carrier is used.

4. The biochemical reaction cassette analysis apparatus according to claim 1, further comprising a communication section which is communicatively connected to an external apparatus via a network and which receives the update information of determination software from the external apparatus.

5. The biochemical reaction cassette analysis apparatus according to claim 2, further comprising a communication section which is communicatively connected to an external apparatus via a network, wherein
the control section acquires version information about appropriate determination software from the storage section based on the probe kind identification information read by the information reading section and sends the acquired determination software version information together with the read probe kind identification information, from the communication section to the external apparatus to request update, and the communication section receives the update information of determination software sent by the external apparatus in response to the update request.

6. The biochemical reaction cassette analysis apparatus according to claim 4, wherein the control section sends the result of analysis of the target substance and information including version information about the determination software used for the analysis and probe kind identification information, from the communication section to the external apparatus.

7. An automatic determination software update system comprising a biochemical reaction cassette analysis apparatus and a determination software provision system which are communicatively connected via a network, wherein
the biochemical reaction cassette analysis apparatus comprises:
a biochemical reaction cassette including a probe carrier on which a probe specifically bindable to a target substance is fixed together with an information medium from which probe kind identification information for identifying the kind of the probe is readable;
a storage section in which determination software for analyzing the target substance is stored in association with version information about the determination software and the probe kind identification information;
an information reading section which reads the probe kind identification information from the information medium; and
a control section which acquires determination software from the storage section based on the probe kind identification information read by the information reading section to use the determination software for analysis of the target substance; and
the determination software provision system comprises:
a determination software information storage section in which determination software corresponding to the kind of the probe is stored in association with version information about the determination software and the probe kind identification information; and
a checking computer which manages update information of determination software stored in the determination software information storage section; wherein
the control section acquires version information about appropriate determination software from the storage section based on the probe kind identification information read by the information reading section and sends the acquired determination software version information together with the read probe kind identification information to the checking computer to request update;
the checking computer acquires, based on the determination software version information and the probe kind identification information received from the control section, appropriate determination software from the determination software information storage section, and, if the acquired determination software version information is newer than the version information received from the control section, sends update information including the acquired determination software, the version information about the determination software and probe kind identification information to the control section; and
the control section updates the information of the determination software stored in the storage section based on the update information received from the checking computer.

8. An automatic determination software update system, comprising a biochemical reaction cassette analysis apparatus and a determination software provision system which are communicatively connected via a network, wherein
the biochemical reaction cassette analysis apparatus comprises:
a biochemical reaction cassette including a probe carrier on which a probe specifically bindable to a target substance is fixed together with an information medium from which probe kind identification information for identifying the kind of the probe is readable;
a storage section in which determination software for analyzing the target substance is stored in association with version information about the determination software and the probe kind identification information;
an information reading section which reads the probe kind identification information from the information medium; and
a control section which acquires determination software from the storage section based on the probe kind identification information read by the information reading section to use the determination software for analysis of the target substance; and
the determination software provision system comprises:
a determination software information storage section in which determination software corresponding to the kind of probe is stored in association with version information about the determination software and the probe kind identification information; and
a checking computer which, when determination software stored in the determination software information storage section is updated, sends update information including the updated determination software, version information about the determination software and probe kind identification information to the biochemical reaction cassette analysis apparatus; wherein
the control section updates the information of the determination software stored in the storage section based on the update information received from the checking computer.

9. The automatic determination software update system according to claim 8, wherein
the control section acquires version information about appropriate determination software from the storage section based on the probe kind identification information read by the information reading section and sends the acquired determination software version information together with the read probe kind identification information to the checking computer to request update; and
the checking computer acquires, based on the determination software version information and the probe kind identification information received from the control section, appropriate determination software from the determination software information storage section, and, if the acquired determination software version information is newer than the version information received from the control section, sends update information including the acquired determination software, the version information about the determination software and probe kind identification information to the control section.

10. The automatic determination software update system according to claim 7, wherein
the determination software provision system further comprises a collected data storage section for storing collected data;
the control section sends the result of analysis of the target substance and information including version information about the determination software used for the analysis and probe kind identification information, to the checking computer as the collected data; and
the checking computer stores the collected data received from the control section in the collected data storage section.

11. An automatic determination software update method performed in a biochemical reaction cassette analysis apparatus having a biochemical reaction cassette including a probe carrier on which a probe specifically bindable to a target substance is fixed together with an information medium from which probe kind identification information for identifying the kind of the probe is readable, the method comprising, as control steps to be performed by a control section:
a first step of storing determination software for analyzing the target substance in a storage section in association with version information about the determination software and the probe kind identification information;
a second step of controlling an information reading section to read the probe kind identification information from the information medium;
a third step of acquiring determination software from the storage section based on the probe kind identification information read by the information reading section to use the determination software for analysis of the target substance; and
a fourth step of, when update information of determination software including the version information and the probe kind identification information is provided from the outside, searching the storage section based on the version information and the probe kind identification information in the provided update information to update information of appropriate determination software.

12. The automatic determination software update method according to claim 11, wherein the fourth step comprises a step of acquiring version information about appropriate determination software from the storage section based on the probe kind identification information read by the information reading section and sending the acquired determination software version information together with the read probe kind identification information to the external apparatus to request update, and receiving the update information of the determination software sent by the external apparatus in response to the update request.

13. The automatic determination software update method according to claim 11, wherein the control step further comprises a step of sending the result of analysis of the target substance and information including version information about the determination software used for the analysis and probe kind identification information, to the external apparatus.

14. An automatic determination software update method performed in a system in which a biochemical reaction cassette analysis apparatus having a biochemical reaction cassette including a probe carrier on which a probe specifically bindable to a target substance is fixed together with an information medium from which probe kind identification information for identifying the kind of the probe is readable is communicatively connected to a determination software provision system via a network, the method comprising, as control steps to be performed by a control section of the biochemical reaction cassette analysis apparatus, the steps of:
storing determination software for analyzing the target substance in a storage section in association with version information about the determination software and the probe kind identification information;
controlling an information reading section to read the probe kind identification information from the information medium;
acquiring determination software from the storage section based on the probe kind identification information read by the information reading section to use the determination software for analysis of the target substance;
acquiring version information about appropriate determination software from the storage section based on the probe kind identification information read by the information reading section and sending the acquired determination software version information together with the read probe kind identification information to the determination software provision system to request update; and
receiving update information of the determination software sent by the determination software provision system in response to the update request, searching the storage section based on version information and probe kind identification information in the received update information, and updating information of appropriate determination software; and
the method comprising, as control steps to be performed by a checking computer of the determination software provision system, the steps of:
storing determination software corresponding to the kind of the probe in a determination software information storage section in association with version information about the determination software and the probe kind identification information; and
acquiring, based on the determination software version information and the probe kind identification information received from the control section, appropriate determination software from the determination software information storage section, and, if the acquired determination software version information is newer than the version information received from the control section, sending update information including the acquired determination software, the version information about the determination software and probe kind identification information to the control section.

15. An automatic determination software update method performed in a system in which a biochemical reaction cassette analysis apparatus having a biochemical reaction cassette including a probe carrier on which a probe specifically bindable to a target substance is fixed together with an information medium from which probe kind identification information for identifying the kind of the probe is readable is communicatively connected to a determination software provision system via a network, the method comprising, as control steps to be performed by a control section of the biochemical reaction cassette analysis apparatus, the steps of:
storing determination software for analyzing the target substance in a storage section in association with version information of the determination software and the probe kind identification information;
controlling an information reading section to read the probe kind identification information from the information medium;
acquiring determination software from the storage section based on the probe kind identification information read by the information reading section to use the determination software for analysis of the target substance; and
updating the information of the determination software stored in the storage section based on the update information received from the determination software provision system; and
the method comprising, as control steps to be performed by a checking computer of the determination software provision system, the steps of:
storing determination software corresponding to the kind of probe in a determination software information storage section in association with version information about the determination software and the probe kind identification information; and
when determination software stored in the determination software information storage section is updated, sending update information including the updated determination software, version information about the determination software and probe kind identification information to the biochemical reaction cassette analysis apparatus.

16. The determination software update method according to claim 14, wherein:
the control step performed by the control section further comprises a step of sending the result of analysis of the target substance and information including version information about the determination software used for the analysis and probe kind identification information, to the checking computer as collected data; and
the control step performed by the checking computer further comprises a step of storing the collected data received from the control section in a collected data storage section.

17. An analysis apparatus for a target substance which is to be used together with a probe carrier on which a probe specifically bindable to a target substance is fixed together with an information medium from which probe kind identification information for identifying the kind of the probe is readable, the analysis apparatus comprising:
(i) a storage section in which determination software for analyzing the target substance is stored in association with version information of the determination software and the probe kind identification information;
(ii) an information reading section which reads the probe kind identification information from the information medium; and
(iii) a control section; wherein
the control section executes the steps of:
acquiring determination software from the storage section based on the probe kind identification information read by the information reading section; and
when update information of determination software including the version information and the probe kind identification information is provided from the outside, searching the storage section based on the version information and the probe kind identification information in the provided update information to update information of appropriate determination software.

18. An analysis apparatus for a target substance which is to be used together with a probe carrier on which a probe specifically bindable to a target substance is fixed together with an information medium from which probe kind identification information for identifying the kind of the probe is readable, the analysis apparatus comprising:
a signal detection section for detecting a signal from the probe carrier;
a storage section in which determination software for analyzing the target substance is stored in association with version information about the determination software and the probe kind identification information;
an information reading section which reads the probe kind identification information from the information medium; and
a control section; wherein
the control section executes the steps of:
(i) causing the signal detection section to detect a signal from the probe carrier provided for reaction with a specimen having a possibility of including the target substance;
(ii) acquiring corresponding determination software from the information medium fixed on the probe carrier mounted on a mounting section, based on the probe kind identification information read by the information reading section;
(iii) requesting update of the determination software acquired at the step (ii); and
(iv) if updated determination software is provided in response to the update request at the step (iii), analyzing the signal detected at the step (i) with the use of the updated determination software.

19. A method of operation of an analysis apparatus for a target substance which is to be used together with a probe carrier on which a probe specifically bindable to a target substance is fixed together with an information medium from which probe kind identification information for identifying the kind of the probe is readable, the analysis apparatus comprising:
a signal detection section for detecting a signal from the probe carrier;
a storage section in which determination software for analyzing the target substance is stored in association with version information about the determination software and the probe kind identification information;
an information reading section which reads the probe kind identification information from the information medium; and
a control section; wherein
the control section executes the steps of:
(i) causing the signal detection section to detect a signal from the probe carrier provided for reaction with a specimen having a possibility of including the target substance;
(ii) acquiring corresponding determination software from the information medium fixed on the probe carrier mounted on a mounting section, based on the probe kind identification information read by the information reading section;
(iii) requesting update of the determination software acquired at the step (ii); and
(iv) if updated determination software is provided in response to the update request at the step (iii), analyzing the signal detected at the step (i) with the use of the updated determination software.
